(19) United States
(12) Patent Application Publication
(10) Pub. No.: US 2024/0189073 A1
(43) Pub. Date: Jun. 13, 2024

(54) INVISIBLE DENTAL BRACE WITH PRESSURE-SENSITIVE SENSORS

(71) Applicant: 3D GLOBAL BIOTECH INC., New Taipei City (TW)

(72) Inventor: Keng-Liang OU, Taipei City (TW)

(73) Assignee: 3D GLOBAL BIOTECH INC., New Taipei City (TW)

(21) Appl. No.: 18/532,774

(22) Filed: Dec. 7, 2023

Related U.S. Application Data
(60) Provisional application No. 63/431,324, filed on Dec. 9, 2022.

(51) Int. Cl. A61C 7/14; A61C 7/18; A61C 7/22; G16H 20/40

(52) U.S. Cl. CPC A61C 7/146; A61C 7/18; A61C 7/22; G16H 20/40

(57) ABSTRACT

The invention relates to an invisible dental brace with pressure-sensitive sensors, comprising: a brace body, at least one pressure-sensitive sensor, and/or a processor. The brace body is designed to receive at least one corresponding tooth of an orthodontic patient and reposition said at least one corresponding tooth from an initial position towards a target position according to a prescribed orthodontic treatment plan. Said at least one pressure-sensitive sensor is configured to generate a sensor signal related to the wearing time of the brace body and a force or pressure applied to said at least one corresponding tooth by the brace body. The sensor signal is then transmitted to a cloud database for further analysis or to the processor for processing to generate a performance of the brace body. Finally, the performance is transmitted to a cloud database for an orthodontic practitioner to modify the prescribed orthodontic treatment plan.

### TECHNICAL FIELD

The present invention relates to the field of dental braces, and more particularly to invisible dental braces with pressure-sensitive sensors for monitoring and recording the wearing condition of an orthodontic patient to help the orthodontic practitioner to determine the causes of the ineffectiveness during the orthodontic treatment.

### BACKGROUND OF THE INVENTION

A large part of the population is having their teeth misaligned in a way that is considered improper for medical or aesthetic reasons, and a misalignment of the teeth is also known as malocclusion. To solve this problem, orthodontic treatment attempts to correct malocclusions and/or improve aesthetics by properly aligning the teeth. Commonly, orthodontic appliances such as dental braces, shell aligners, and the like are applied to the patient's teeth by an orthodontic practitioner to properly align the teeth. The dental braces can be configured to exert force on the teeth of an orthodontic patient to achieve desired tooth movements according to a prescribed plan of orthodontic treatment.

Recently, with the development in the field of material engineering and rapid prototyping techniques, invisible dental braces have been developed and are widely accepted between orthodontic patients and orthodontic practitioners for several reasons, including free of dietary restriction, invisible appearance, and more comfortable and less pain.

Despite the obvious benefits of the usage of an invisible dental brace, if the process of orthodontic treatment is not as effective as expected, the orthodontic practitioner is unable to determine whether it is caused by the failure of the invisible dental brace or by the intermittent wearing conditions of the orthodontic patients.

Therefore, there is an indispensable need for developing a method to monitor the wearing condition of the invisible dental brace used by the orthodontic patient. However, practically the forces applied to a patient's teeth by an invisible dental brace may differ from the desired forces for the prescribed treatment plan and may result in incomplete or undesirable tooth movements. In other words, the amount and duration of the force applied by the invisible dental brace must be controlled and monitored throughout the orthodontic treatment to avoid undesirable effects, such as tooth root resorption, alveolar bone remodelling, and pain or discomfort caused by the invisible dental brace.

Accordingly, inventors of the present application have made a tremendous effort to make inventive research and eventually provide an invisible dental brace with pressure-sensitive sensors for monitoring and recording the performance of the invisible dental brace and the wearing condition of an orthodontic patient to help the orthodontic practitioner to determine the causes of the ineffectiveness or failure during the orthodontic treatment.

### SUMMARY OF THE INVENTION

The prime objective of the present invention is to disclose an invisible dental brace with pressure-sensitive sensors, comprising a brace body, at least one pressure-sensitive sensor, and/or a processor. The brace body is designed to comprise a least one cavity for receiving at least one corresponding tooth of an orthodontic patient and repositioning said at least one corresponding tooth from an initial position towards a target position according to the prescribed plan of orthodontic treatment. Said at least one pressure-sensitive sensor is arranged on a buccal and/or lingual side of said at least one cavity, i.e. said at least one pressure-sensitive sensor is attached to the wall on the buccal and/or lingual side of said at least one cavity. Moreover, said at least one pressure-sensitive sensor is configured to generate a sensor signal related to the wearing time of the brace body and a force or pressure applied to said at least one corresponding tooth of an orthodontic patient by the brace body. The processor is electrically connected to said at least one pressure-sensitive sensor and is configured to receive, store, and process the sensor signal. In addition, the sensor signal can either be transmitted directly from said at least one pressure-sensitive sensor to a cloud database for further analysis or be transmitted to the processor for processing to evaluate the performance of said brace body. The performance of said brace body generated by the processor is then transferred to a cloud database for an orthodontic practitioner to adjust the prescribed plan of orthodontic treatment.

For achieving the prime objective mentioned above, the present invention provides an embodiment of an invisible dental brace with pressure-sensitive sensors, comprising:
a brace body comprising a least one cavity for receiving at least one corresponding tooth of an orthodontic patient and repositioning said at least one corresponding tooth from an initial position towards a target position according to a prescribed plan of orthodontic treatment;
at least one pressure-sensitive sensor arranged on a buccal and/or lingual side of said at least one cavity, wherein said at least one pressure-sensitive sensor is configured to generate a sensor signal related to the wearing time of the brace body and a force or pressure produced between each cavity of said at least one cavity and each tooth of said at least one corresponding tooth and/or configured to transmit the sensor signal wirelessly to a cloud database for further analysis and for an orthodontic practitioner to modify the prescribed plan of orthodontic treatment; and/or
a processor coupled to the brace body and configured to receive, store, and process the sensor signal, wherein the processor generates a performance of the brace body based on the sensor signal and transmitted the performance wirelessly to the cloud database for an orthodontic practitioner to modify the prescribed plan of orthodontic treatment.

In one embodiment, the invisible dental brace with pressure-sensitive sensors further comprises a power source, memory and wireless communication circuit. The power source may be a flexible, thin and/or printed battery, such as a zinc-carbon flexible battery, a zinc-manganese dioxide printed flexible battery, or a solid-state thin-film lithium phosphorus oxynitride battery, for providing power to said at least one pressure-sensitive sensor and said processor. The usage of a flexible, thin and/or printed battery is beneficial for reducing the overall size of said invisible dental brace with pressure-sensitive sensors. Furthermore, the memory may be used to store the sensor signal and the performance generated respectively by said at least one pressure-sensitive sensor and said processor. The memory comprises RAM such as SRAM or DRAM; ROM such as EPROM, PROM, or MROM; or hybrid memory such as EEPROM, flash, or NVRAM.

In one embodiment, said at least one pressure-sensitive sensor may be a force-sensitive sensor and comprises a force-sensitive film, a pressure-sensitive film, a resistive film, a capacitive film or a piezoresistive pressure sensor.

In another embodiment, said at least one pressure-sensitive sensor is configured to directly transmit the sensor signal to a cloud database wirelessly by said wireless communication circuit to a cloud database through any existing wireless communication technology for further analysis, wherein said wireless communication technology, including WiFi, Bluetooth, WiMax, and cellular network.

In a further embodiment, the sensor signal further comprises tooth movement data, tooth position data or tooth identification data, and the processor is configured to evaluate the performance of the brace body by using said sensor signal generated by and received from said at least one pressure-sensitive sensor.

In another further embodiment, the performance determined and evaluated by the processor is then transmitted wirelessly by said wireless communication circuit to a cloud database through any existing wireless communication technology, wherein the wireless communication technology comprises WiFi, Bluetooth, WiMax or cellular network.

In addition, the cloud database can be a remote device of any computing device or system, such as a personal computer, laptop, tablet, mobile device, wearable device, etc.

### BRIEF DESCRIPTION OF THE DRAWINGS

The invention, as well as a preferred mode of use and advantages thereof, will be best understood by referring to the following detailed description of an illustrative embodiment in conjunction with the accompanying drawings, wherein:

FIG. 1 is a schematic illustration of an invisible dental brace with a pressure-sensitive sensor according to an embodiment of the present invention;

FIG. 2 is a schematic illustration of an invisible dental brace with a pressure-sensitive sensor worn by an orthodontic patient according to an embodiment of the present invention;

FIG. 3 is a schematic illustration of a sensor signal transmitted from a pressure-sensitive sensor to a cloud database; and

FIG. 4 shows a flowchart of a method of orthodontic treatment using an invisible dental brace with pressure-sensitive sensors according to the present invention.

### DETAILED DESCRIPTION OF THE PREFERRED EMBODIMENTS

To better illustrate the advantages of the invisible dental brace with pressure-sensitive sensors according to the present invention and its contributions to the art, preferred embodiments of the present invention will be described in detail concerning the attached drawings hereafter.

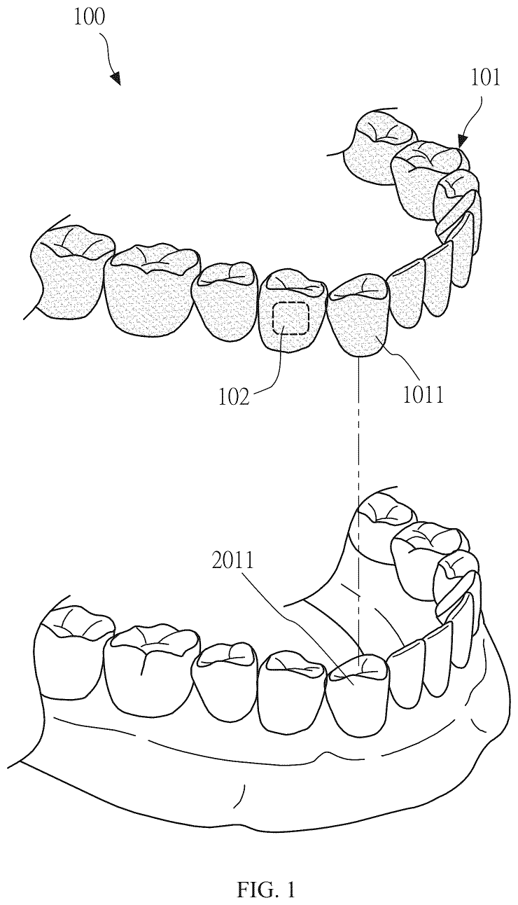

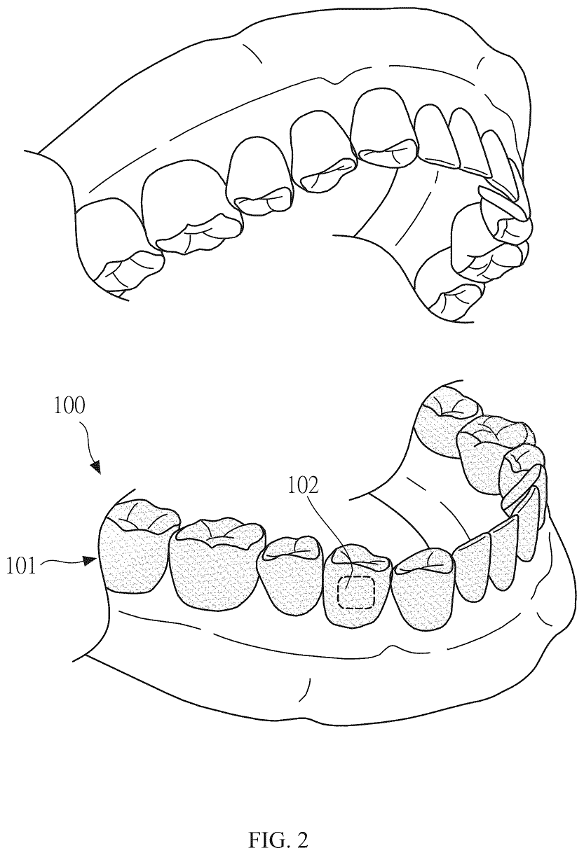

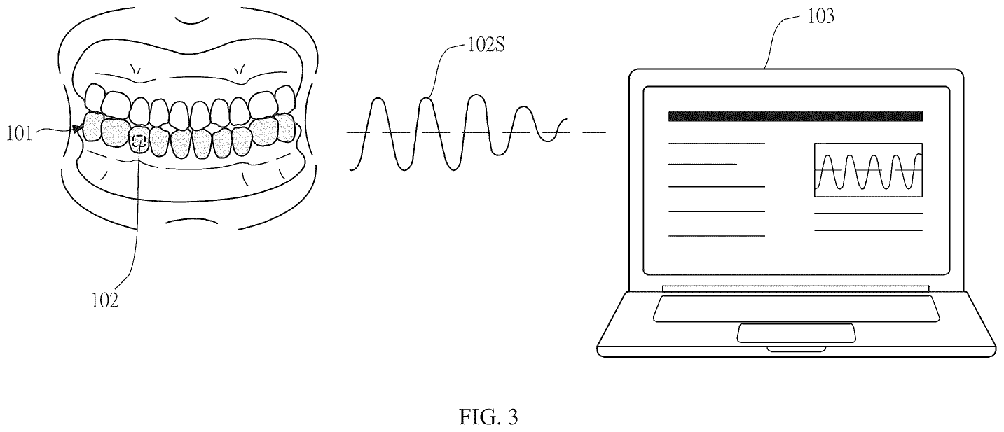

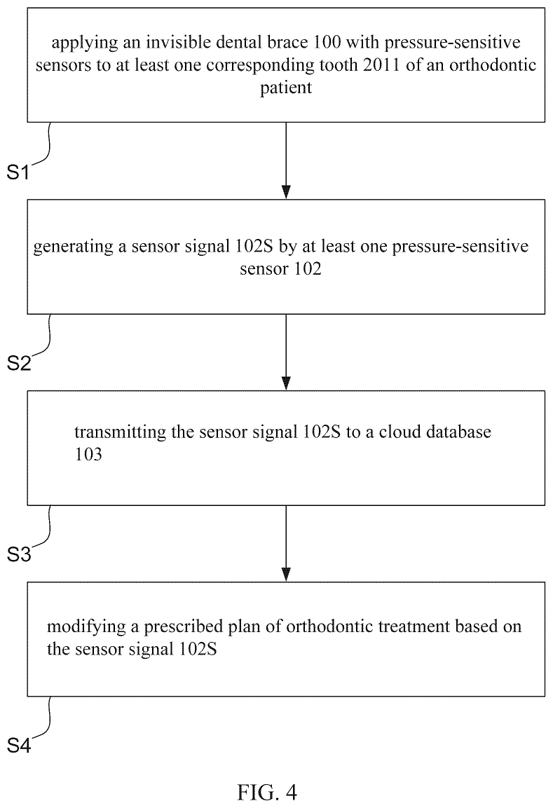

FIG. 1 is an illustrative schematic diagram that shows an invisible dental brace with a pressure-sensitive sensor according to the present invention, wherein the pressure-sensitive sensor is attached to the wall on the buccal side of one of the cavities of a brace body. FIG. 2 illustrates an invisible dental brace with a pressure-sensitive sensor worn by an orthodontic patient according to the present invention. FIG. 3 shows a sensor signal generated by a pressure-sensitive sensor is transmitted wirelessly from a pressure-sensitive sensor to a cloud database. FIG. 4 shows a flowchart of an orthodontic treatment using an invisible dental brace with pressure-sensitive sensors according to the present invention.

According to FIG. 1 and FIG. 2, an invisible dental brace 100 with pressure-sensitive sensors comprises a brace body 101 and at least one pressure-sensitive sensor 102. The brace body 101 is designed to comprise a least one cavity 1011 for receiving at least one corresponding tooth 2011 of an orthodontic patient and repositioning said at least one corresponding tooth 2011 from an initial position towards a target position according to the prescribed plan of orthodontic treatment. Said at least one pressure-sensitive sensor 102 is arranged on a buccal and/or lingual side of said at least one cavity 1011. In other words, said at least one pressure-sensitive sensor 102 is attached to the wall on the buccal and/or lingual side of said at least one cavity 1011. In FIG. 2, the exemplary embodiment shows that the invisible dental brace 100 with pressure-sensitive sensors is worn in the bottom row of teeth and said at least one pressure-sensitive sensor 102 is arranged on a buccal side of said at least one cavity 1011.

According to FIG. 3, said at least one pressure-sensitive sensor 102 is configured to generate a sensor signal 102S related to the wearing time of the brace body and a force or pressure applied to said at least one corresponding tooth 2011 of an orthodontic patient by the brace body 101. Then the sensor signal 102S generated by said at least one pressure-sensitive sensor 102 can be transmitted wirelessly from said at least one pressure-sensitive sensor 102 to a cloud database 103 for further analysis by an orthodontic practitioner to modify the prescribed plan of orthodontic treatment.

In one embodiment, the invisible dental brace 100 with a pressure-sensitive sensor according to the present invention can further comprise a processor (not shown). The processor is electrically connected to said at least one pressure-sensitive sensor 102 and is configured to receive, store, and process the sensor signal 102S generated by said at least one pressure-sensitive sensor 102. After the processor received the sensor signal 102S, the sensor signal 102S is processed by the processor to produce a performance of the brace body 101 and then the performance is transmitted wirelessly to a cloud database 103 for an orthodontic practitioner to adjust the prescribed plan of orthodontic treatment.

In other words, the sensor signal 102S can either be transmitted directly from said at least one pressure-sensitive sensor 102 to a cloud database 103 for further analysis or be transmitted to the processor for processing to evaluate the performance of said brace body 101.

In one embodiment, the sensor signal 102S comprises tooth movement data, tooth position data or tooth identification data and can be transmitted wirelessly to a cloud database 103 through any existing wireless communication technology, wherein the wireless communication technology comprises WiFi, Bluetooth, WiMax or cellular network. In addition, the cloud database 103 can be a remote device of any computing device or system, such as a personal computer, laptop, tablet, mobile device, wearable device, etc.

In a further embodiment, the invisible dental brace 100 with pressure-sensitive sensors may comprise a power source and memory (not shown). The power source may be a flexible, thin and/or printed battery, such as a zinc-carbon flexible battery, a zinc-manganese dioxide printed flexible battery, or a solid-state thin-film lithium phosphorus oxynitride battery, for providing power to said at least one pressure-sensitive sensor 102 and said processor. The usage of a flexible, thin and/or printed battery is advantageous for reducing the overall size of said invisible dental brace 100 with pressure-sensitive sensors. Furthermore, the memory may be used to store the sensor signal 1028 and the performance generated respectively by said at least one pressure-sensitive sensor 102 and said processor. The memory comprises RAM such as SRAM or DRAM; ROM such as EPROM, PROM, or MROM; or hybrid memory such as EEPROM, flash, or NVRAM.

According to FIG. 4, a method of orthodontic treatment using an invisible dental brace 100 with pressure-sensitive sensors according to the present invention comprises the following steps:

S1: applying an invisible dental brace 100 with pressure-sensitive sensors to at least one corresponding tooth 2011 of an orthodontic patient;

S2: generating a sensor signal 102S associated with the wearing time of the brace body 101, a force or pressure applied to said at least one corresponding tooth 2011 of the orthodontic patient, tooth movement data, tooth position data or tooth identification data by at least one pressure-sensitive sensor 102;

S3: transmitting the sensor signal 102S to a cloud database 103 from said at least one pressure-sensitive sensor 102 for further analysis; and

S4: modifying a prescribed plan of orthodontic treatment based on the sensor signals 102S.

In a nutshell, the above descriptions have thoroughly introduced the invisible dental brace with pressure-sensitive sensors according to the present invention. The above descriptions are made on embodiments of the present invention; however, the embodiments are not intended to limit the scope of the present invention, and all equivalent implementations or alterations within the spirit of the present invention still fall within the scope of the present invention.

For another embodiment, an invisible dental brace comprising a brace body, at least one pressure-sensitive sensor, and a processor, wherein the brace body is made from a transparent material and the pressure-sensitive sensors are strategically placed within the brace body to detect the pressure exerted by the teeth during movement.

Wherein the brace body is made from a transparent material that blends seamlessly with the natural color of the teeth. The pressure-sensitive sensors are capable of accurately measuring the force applied to the teeth. The processor is integrated into the brace body and the processor is wirelessly connected to an external device.

The processor analyzes the data collected by the pressure-sensitive sensors to determine the effectiveness of the orthodontic treatment and provides recommendations for adjustments if necessary. The recommendations for adjustments are communicated to the user and the orthodontist.

For another embodiment, an invisible dental brace system for tooth repositioning, comprising: a brace body comprising a base portion and a plurality of brackets, each bracket being adapted to receive and engage a tooth; wherein the base portion is configured to be securely attached to the patient's teeth; wherein the brackets are movably connected to the base portion.

Wherein the base portion further comprises a plurality of attachment elements for securely attaching the base portion to the patient's teeth. Each bracket further comprises a slot for receiving a wire or archwire. The brackets are adjustably connected to the base portion, allowing for controlled tooth movement.

The invisible dental brace system further comprising a plurality of elastic bands for connecting the brackets to the base portion, providing additional force for tooth repositioning, a plurality of auxiliary attachments for connecting the brackets to the base portion, allowing for customized tooth movement, a plurality of adjustment mechanisms for fine-tuning the tooth movement, and a plurality of indicators for monitoring the progress of tooth repositioning.

Wherein the auxiliary attachments comprise hooks, loops, or clips for receiving additional orthodontic appliances.

Wherein the adjustment mechanisms comprise screws, springs, or ratchets.

A pressure-sensitive sensor is a sensor that can measure pressure. Common pressure-sensitive sensors include strain gauges, capacitive sensors, optical sensors, and magnetostrictive sensors. The pressure-sensitive sensor can be used to monitor the force or pressure applied by the orthodontic appliance to the teeth. The sensor should have the following characteristics: High sensitivity, which can accurately measure small changes in pressure; Good stability, which can be used for a long time without failure; High reliability, which can ensure normal operation in harsh environments.

A processor is an electronic device that can receive, store, and process data. In my invention, the processor can be used to receive, store, and process the sensor signals generated by the pressure-sensitive sensor. The processor should have the following characteristics: Strong computational ability, which can quickly process complex data; Large storage capacity, which can store a large amount of data; Low power consumption, which can extend battery life.

A wireless communication circuit is an electronic device that can wirelessly transmit data. In my invention, the wireless communication circuit can be used to transmit the data generated by the processor to the cloud database. The wireless communication circuit should have the following characteristics: Long communication distance, which can cover a larger area; Fast transmission speed, which can quickly transmit a large amount of data; Strong anti-interference ability, which can work normally in harsh environments.

A power supply is a device that can provide power to electronic devices. In my invention, the power supply can be used to power the pressure-sensitive sensor, processor, and wireless communication circuit. The power supply should have the following characteristics: High power density, which can provide enough power for electronic devices; Long life, which can be used for a long time without damage; Safe and reliable, which can prevent battery explosions or fires.

Memory is an electronic device that can store data. In my invention, the memory can be used to store the data generated by the pressure-sensitive sensor, processor, and wireless communication circuit. The memory should have the following characteristics: Large capacity, which can store a large amount of data; Fast read and write speed, which can quickly read and write data; High reliability, which can ensure data security.

A cloud database is a database stored in the cloud. In my invention, the cloud database can be used to store the data generated by the processor. The cloud database should have the following characteristics: Large capacity, which can store a large amount of data; High security, which can prevent data from being stolen or tampered with; Good scalability, which can be expanded as the amount of data increases.

According to the above technical description, the present invention can be used in the following specific application scenarios:

Wearing time monitoring: It can be used to monitor whether the patient is wearing the orthodontic appliance according to the regulations. If the patient's wearing time is insufficient, the patient can be reminded to wear it in time.

Force monitoring: It can be used to monitor the force applied by the orthodontic appliance to the teeth. If the force is too large, the orthodontist can be reminded to adjust the design of the orthodontic appliance.

Orthodontic effect evaluation: It can be used to evaluate the orthodontic effect of the orthodontic appliance. If the orthodontic effect is not good, the orthodontist can be reminded to adjust the orthodontic treatment plan.

In aligners, pressure-sensitive sensors are a key technology used to evaluate the impact of aligners on each tooth. The application and operation flow of this sensor can be described in detail as follows:

Step 1: Start of wear time: When the patient starts wearing aligners, the pressure-sensitive sensors also start working. They immediately sense the forces and pressures interacting with the teeth.

Step 2: Generation of sensor signals: The sensors start generating sensor signals related to the wearing time and the pressure applied to the teeth in each cavity. This could be a series of data points, representing the pressure status of each corresponding tooth over time.

Step 3: Connection to the processor: The generated sensor signals are transmitted to the processor, which is the electronic device connected to the sensors. This connection is typically achieved through flexible, thin, and/or printed circuits.

Step 4: Data storage: The processor stores these data in memory for subsequent processing and analysis. This helps to record the pressure status at each time point during the orthodontic process.

Step 5: Generation of performance: Based on the sensor data, the processor generates information about the performance of the aligners. This could include the degree of adjustment to each corresponding tooth, changes in pressure, and so on.

Step 6: Wireless transmission: The processor can wirelessly transmit these performance data to a cloud database through wireless communication circuits. Such transmission is typically real-time, enabling real-time monitoring of the treatment.

Data transmission process between pressure-sensitive sensors and cloud database:Data generation by pressure-sensitive sensors: Pressure-sensitive sensors sense changes in pressure interacting with the teeth, and generate data related to wearing time and the pressure applied to the teeth in each cavity in real time.

Data connection to processor: These data are connected to the processor, which is the device electrically connected to the sensors, through flexible, thin, and/or printed circuits.

Data analysis and performance data generation by processor: The processor analyzes sensor data and generates information about the performance of the aligners, such as the adjustment status of the teeth, changes in pressure, and so on.

Data storage: These performance data are stored in the memory of the processor, which could be RAM (SRAM or DRAM), ROM (EPROM, PROM, or MROM), or mixed memory (EEPROM, flash, or NVRAM).

Power supply: The operation of the processor and sensors requires a power supply. Flexible, thin, and/or printed batteries (such as zinc-carbon flexible batteries, zinc-manganese dioxide printed flexible batteries, or solid-state thin-film lithium-phosphorus-nitrogen oxide batteries) are typically used for this application, which helps to reduce overall size.

Wireless communication circuit: The processor is connected to a wireless communication circuit, which is implemented through technologies such as WiFi, Bluetooth, WiMax, or cellular networks. This part supports wireless data transmission.

Data transmission to cloud database: The wireless communication circuit transmits the performance data generated by the processor to the cloud database through the selected wireless technology. This could be real-time, enabling real-time monitoring of the treatment.

Cloud reception and storage: The cloud database receives and stores data from the processor. Such a cloud database could be a remote device, such as a personal computer, laptop, tablet, mobile device, and so on.

Further analysis and diagnosis: In the cloud, the data can be further analyzed, including analysis of tooth movement data, location data, or tooth identification data. This provides orthodontists with more information to assess the effectiveness of the aligners and make adjustments.

What is claimed is:

1. An invisible dental brace with pressure-sensitive sensors comprising:
a brace body comprising at least one cavity for receiving at least one corresponding tooth of an orthodontic patient and repositioning said at least one corresponding tooth from an initial position towards a target position according to a prescribed plan of orthodontic treatment;
at least one pressure-sensitive sensor arranged on a buccal and/or lingual side of said at least one cavity, wherein said at least one pressure-sensitive sensor is configured to generate a sensor signal related to wearing time of the brace body and a force or pressure produced between each cavity of said at least one cavity and each tooth of said at least one corresponding tooth and/or configured to transmit the sensor signal (102S) wirelessly to a cloud database (103) for further analysis and for an orthodontic practitioner to modify the prescribed plan of orthodontic treatment; and/or
a processor coupled to the brace body (101) and configured to receive, store, and process the sensor signal (102S), wherein the processor generates a performance of the brace body (101) based on the sensor signal (102S) and transmitted the performance wirelessly to the cloud database (103) for an orthodontic practitioner to modify the prescribed plan of orthodontic treatment.

2. An invisible dental brace comprising a brace body, at least one pressure-sensitive sensor, and a processor, wherein the brace body is made from a transparent material and the pressure-sensitive sensor(s) are strategically placed within the brace body to detect the pressure exerted by the teeth during movement.

3. The invisible dental brace of claim 2, wherein the brace body is made from a transparent material that blends seamlessly with the natural color of the teeth.

4. The invisible dental brace of claim 2, wherein the pressure-sensitive sensors are capable of accurately measuring the force applied to the teeth.

5. The invisible dental brace of claim 2, wherein the processor is integrated into the brace body.

6. The invisible dental brace of claim 2, wherein the processor is wirelessly connected to an external device.

7. The invisible dental brace of claim 2, wherein the processor analyzes the data collected by the pressure-sensitive sensors to determine the effectiveness of the orthodontic treatment and provides recommendations for adjustments if necessary.

8. The invisible dental brace of claim 7, wherein the recommendations for adjustments are communicated to the user and the orthodontist.

9. An invisible dental brace system for tooth repositioning, comprising:
a brace body comprising a base portion and a plurality of brackets, each bracket being adapted to receive and engage a tooth;
wherein the base portion is configured to be securely attached to the patient's teeth;
wherein the brackets are movably connected to the base portion.

10. The invisible dental brace system of claim 9, wherein the base portion further comprises a plurality of attachment elements for securely attaching the base portion to the patient's teeth.

11. The invisible dental brace system of claim 9, wherein each bracket further comprises a slot for receiving a wire or archwire.

12. The invisible dental brace system of claim 9, wherein the brackets are adjustably connected to the base portion, allowing for controlled tooth movement.

13. The invisible dental brace system of claim 9, further comprising a plurality of elastic bands for connecting the brackets to the base portion, providing additional force for tooth repositioning.

14. The invisible dental brace system of claim 9, further comprising a plurality of auxiliary attachments for connecting the brackets to the base portion, allowing for customized tooth movement.

15. The invisible dental brace system of claim 14, wherein the auxiliary attachments comprise hooks, loops, or clips for receiving additional orthodontic appliances.

16. The invisible dental brace system of claim 9, further comprising a plurality of adjustment mechanisms for fine-tuning the tooth movement.

17. The invisible dental brace system of claim 16, wherein the adjustment mechanisms comprise screws, springs, or ratchets.

18. The invisible dental brace system of claim 9, further comprising a plurality of indicators for monitoring the progress of tooth repositioning.
